(19) United States
(12) Patent Application Publication
(10) Pub. No.: US 2012/0244592 A1
(43) Pub. Date: Sep. 27, 2012

(54) METHOD FOR AMPLIFYING THE PENTOSE PHOSPHATE PATHWAY IN YEAST STRAINS, THE RESULTING YEAST AND USES THEREOF

(76) Inventors: Sylvie Dequin, Montpellier (FR); Anne Ortiz-Julien, Cagnac sur Garonne (FR); Axelle Cadiere, Beauvoisin (FR)

(21) Appl. No.: 13/510,442

(22) PCT Filed: Nov. 18, 2010
(86) PCT No.: PCT/IB2010/055257

§ 371 Date: May 17, 2012

(30) Foreign Application Priority Data
Nov. 20, 2009 (FR) 09/05585

(51) Int. Cl. C12N 1/19; C12P 1/02

(52) U.S. Cl. 435/171; 435/254.2; 435/254.21

(57) ABSTRACT

The invention relates to a method for obtaining mutant yeast strains having an amplified PPP, characterised in that it comprises the culture of the strains on a substrate containing a gluconic acid salt.

The subject of the invention is a method for increasing the pentose phosphate pathway (PPP) flux and the resulting yeasts. The invention is also directed toward the uses of these yeasts, in particular in the fermentation industries.

Since the PPP is the center of carbon and redox metabolism in yeast, amplification of this pathway is of interest in terms of resulting in considerable metabolic remodeling and thus conferring new properties on the yeast.

Obtaining a yeast strain having an amplified PPP can be envisioned through various strategies.

Numerous approaches have been developed over the past few years with the aim of obtaining S. cerevisiae strains capable of fermenting pentoses. Thus, S. cerevisiae strains capable of fermenting xylose to ethanol have been obtained through the expression of heterologous genes allowing its assimilation (Eliasson et al., 2000). However, the formation of ethanol by these strains remains limited because of the secretion of a part of the xylose consumed, in the form of xylitol. The xylitol accumulation could come from the limitation of the flux through the nonoxidative part of the PPP in S. cerevisiae. Indeed, studies on mutants with a better capacity for metabolizing xylose have shown an increase in the activity of their transaldolase or their transketolase, or both (Becker et al., 2003; Sonderegger et al., 2005; Pitkanen et al., 2005). Furthermore, the deletion of one of the two genes (ZWF1, GND1) encoding the NADPH enzymes dependent on the oxidative segment makes it possible to also reduce the xylitol production yield and to increase the ethanol yield (Eliasson et al., 2000; Jeppsson et al., 2002).

The selection of mutants by directed evolution constitutes an alternative to metabolic engineering approaches. This approach is based on the selection of variants, obtained by maintaining a strain for numerous generations under selective conditions so as to force adaptation. This selection principle was chosen by the inventors who, surprisingly, have succeeded in selecting variants having accumulated mutations allowing a significant amplification of the PPP. These mutations make it possible to degrade more gluconate and confer new, completely unexpected properties on the strains (reduction in acetate production, increase in that of esters, reduced nitrogen requirements).

The objective of the invention is therefore to provide a method for obtaining yeast mutants in which the PPP is amplified compared with a parental strain.

The invention is also directed toward providing yeast mutants of which the metabolism, unexpectedly, has been remodeled compared with the parental strains, following amplification of the PPP.

The invention is also directed toward taking advantage of the properties of these yeasts, in particular in the fermentation industries.

The method for obtaining mutant yeast strains according to the invention is characterized in that it comprises culturing the strains on a substrate based on a gluconic acid derivative, more particularly a gluconic acid salt (such a salt being designated hereinafter by the abbreviation “gluconate”).

Advantageously, the gluconate is incorporated into the PPP at the level of the 3rd stage.

The culturing of the strains is carried out in order to allow them to adapt under unfavorable culture conditions, over at least 10 generations, to an incubation temperature of from 16° C. to 32° C., preferably of 28° C., with shaking.

The mutants which have accumulated mutations allowing amplification of the PPP are advantageously selected by means of a growth test, and those having an optical density (OD) greater than at least 1.3 times that of the parental strain are recovered.

The invention is also directed toward the yeast strain mutants which have an enhanced PPP compared with a parental strain.

These mutants are characterized in that they are capable of being obtained by means of a method of culture comprising the steps
of adapting a yeast strain, over at least 10 generations, using as sole carbon source a substrate based on gluconate,
of selecting the mutants having accumulated mutations allowing an amplification of the PPP, having a final OD at least 1.3 times greater than that of the parental strain not subjected to the above adaptation conditions.

More especially, the invention is directed toward the yeast strain mutants having, compared with a parental strain, an enhanced PPP, a higher fermentation rate and, consequently, a faster glucose consumption, and also a reduction in acetate production.

Advantageously, the mutants of the invention are characterized by a high production of aromatic compounds, such as isoamyl alcohol, isoamyl acetate or 2-phenylethanol.

The yeast strains preferably belong to the Saccharomyces genus, in particular to the species cerevisiae or bayanus, or to non-Saccharomyces genera.

Preferred strains belong to the Saccharomyces genus and comprise, in particular, the species Saccharomyces cerevisiae, Saccharomyces bayanus, Saccharomyces uvarum and Saccharomyces kudriavzevii.

Other preferred strains belong to the non-Saccharomyces genus.

The invention is also directed toward the hybrids of the strains defined above.

The invention is in particular directed toward the adapted Saccharomyces cerevisiae strain deposited at the Collection Nationale de Culture de Microorganismes (CNCM) [French National Microorganism Culture Collection], 25 rue du Dr Roux, Paris, for the purposes of a deposit according to the Treaty of Budapest, under No. CNCM I-4216, on Jul. 29, 2009.

The amplification of the PPP in the mutant strains of the invention allows, in particular:
a decrease in ethanol production,
a decrease in acetate production,
a modification of the production of certain aroma compounds,
an increase in fermentation rate.

These properties are of major interest for various uses of the yeast, in particular in the fermentation industries.

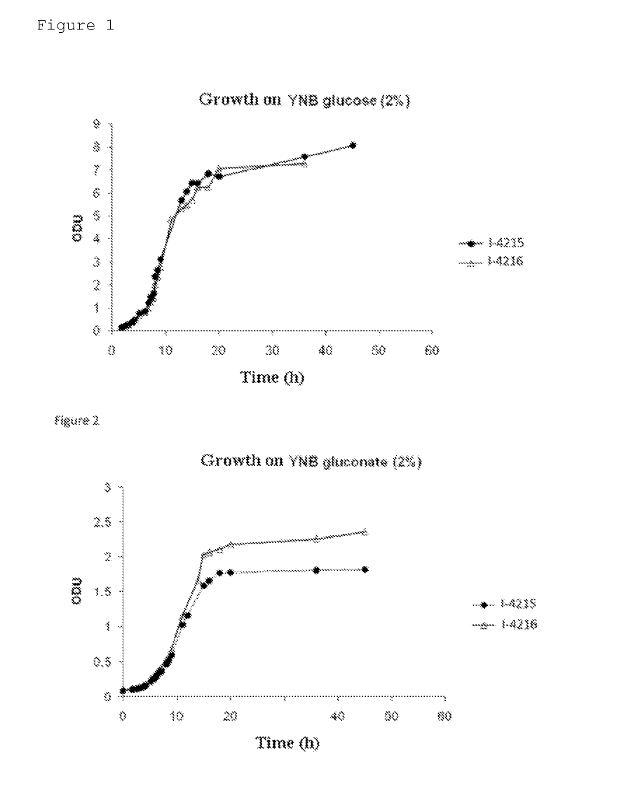

Other features and advantages of the invention are given in the examples which follow, in which reference is made to FIGS. 1 to 5, which represent, respectively,

FIGS. 1A and 1B: the results of growth tests for a strain resulting from an adaptation on gluconate (I-4216) and for the parental strain (I-4215), on YNB glucose (2%) medium (FIG. 1A) and on YNB gluconate (2%) medium (FIG. 1B);

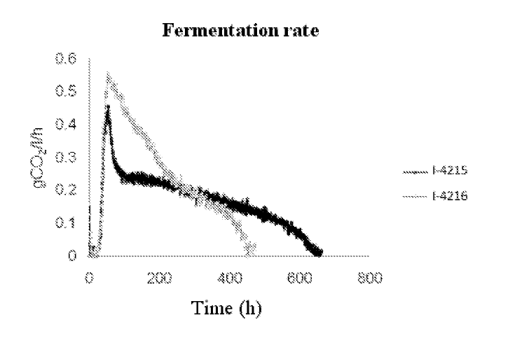

FIG. 2, the fermentation rate of these strains;

FIG. 3, the results relating to acetate production with a strain of the invention and the parental strain;

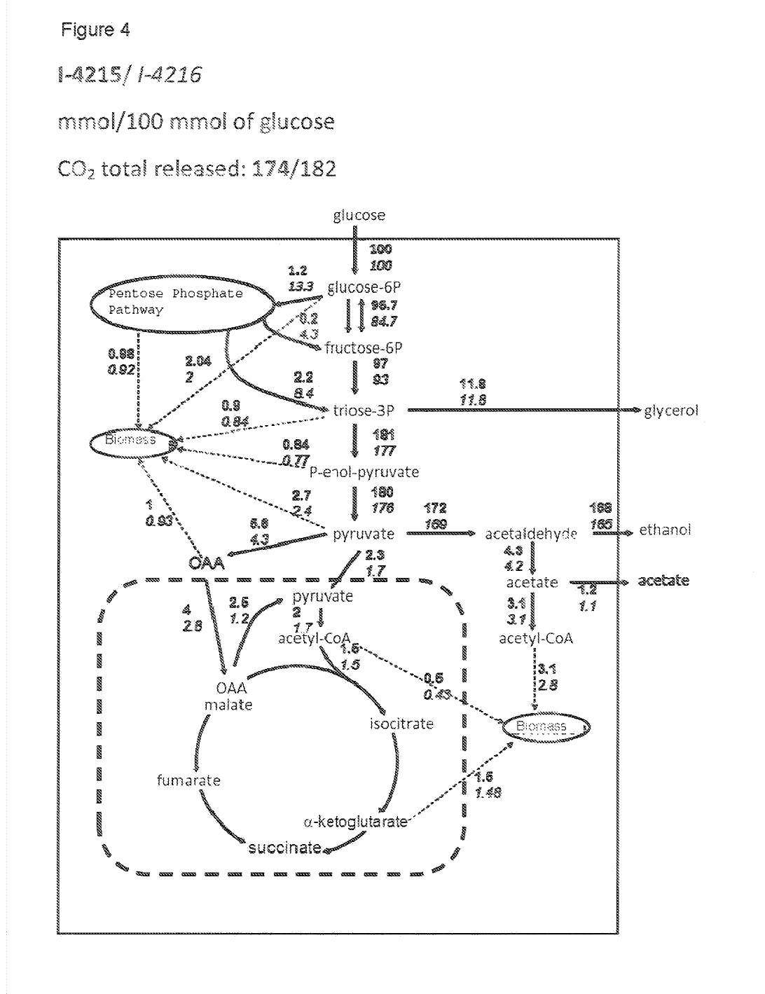

FIG. 4, the distribution of the metabolic fluxes in a strain of the invention and in a parental strain, and

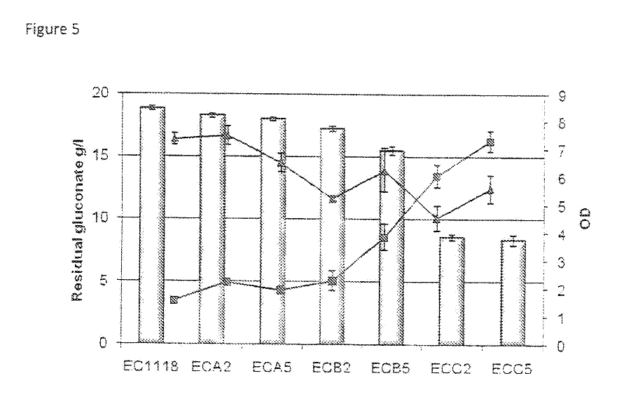

FIG. 5, the histograms representing the residual gluconate in the medium.

Directed Adaptation and Culture Mode

The I-4215 industrial strain was cultured in 13 ml tubes containing 5 ml of YNB gluconate medium [0.67% DIFCO yeast nitrogen base, and 2% gluconate (Sigma)]. Two cultures were performed in parallel. The cultures are inoculated at an optical density (OD) of 0.1, from an overnight preculture on YEPD (1% Bacto Yeast Extract (Difco), 2% Bacto Peptone (Difco), 2% glucose). The cells are centrifuged and washed with sterile water in order to remove any trace of glucose. The culture is performed for numerous generations, by diluting the cells by transfer into a new medium every 3 generations (OD=0.8). The cultures are incubated at 28° C. with shaking (250 rpm). Since growth on this substrate is very slow, 8 days are necessary in order to reach an OD of 0.8.

Analysis of the Adapted Strains

At regular intervals, 200 μl of cultures are plated out on a dish containing YEPD medium, in order to carry out growth tests for demonstrating a change in the strains. To this effect, the growth of the strains resulting from the adaptation is compared with that of the I-4215 parental strain, on YNB gluconate (2%) and on YNB glucose (2%). The cultures are inoculated at OD 0.1, from an approximately 14 h preculture on YEPD. The cultures are performed in 250 ml Erlenmeyer flasks containing 50 ml of medium and incubated at 28° C. with shaking (250 rpm).

After 23, 37 and 79 transfers corresponding to approximately 70, 180 and 240 generations, strains resulting from the two adaptations carried out in parallel were isolated on the basis of change in YNB gluconate (2%) growth capacities. These strains are called ECA2 and I-4216 for the selection carried out at 70 generations, ECB2 and ECB5 for the selection carried out at 180 generations, and ECC2 and ECC5 for that carried out at 240 generations (FIG. 1).

Their capacity for consuming gluconate is represented in FIG. 5 (black squares: final biomass on YNB gluconate; black triangles: final biomass on YNB glucose). The more the adaptation is prolonged, the more the capacity for consuming gluconate increases. On the other hand, the capacity of these strains for multiplying on glucose decreases the more the adaptation is prolonged.

All these strains were tested under enological fermentation conditions on MS 70 containing 240 g/l of glucose at 18° C. The I-4216 strain exhibits the best fermentative capacities (fermentation time reduced by 30%), whereas the ECB2 and ECC2 strains do not finish their fermentation. The ECA2, ECB5 and ECC5 strains exhibit an intermediate fermentative phenotype between I-4216 and I-4215. A detailed comparative analysis of the properties of the I-4216 and I-4215 strains was then carried out.

No difference in growth was observed on YNB glucose between this strain and I-4215 (FIG. 2).

On the other hand, on YNB gluconate, the final OD reached by I-4216 is 1.3 times greater than that reached by I-4215 (FIG. 2).

Fermentative Properties of the Adapted Strain I-4216

Culture Conditions

The fermentations were carried out in 1.2-liter reactors (SGI, France) with a reaction volume of 1 liter. MS medium was used for the preculture and the culture. It is a synthetic medium which simulates a standard grape must. MS medium contains 20% glucose, 6 g/l of malic acid, 6 g/l of citric acid and 100 mg/l of nitrogen, in the form of NH₄Cl (79 mg/l) and of amino acids (28 mg/l). The pH of the MS medium is adjusted to 3.3 with a 32% NaOH solution. Anerobiosis factors, ergosterol (7.5 mg/l), oleic acid (2.5 mg/l) and Tween 80 (0.21 g/l) are added. The precultures were performed in 250 ml Erlenmeyer flasks containing 50 ml of YEPD medium at 28° C. with shaking (150 rpm) for 8 h. These precultures were inoculated at 0.1 ODU/ml from a preculture on YEPD carried out for approximately 14 h. The reactors were inoculated from the precultures, at 0.5 million cells per ml, and kept at a constant temperature of 18° C. with continuous shaking (300 rpm).

Analytical Methods

The growth was monitored by counting the number of cells on a Coulter Counter apparatus (ZBI) using an aliquot fraction of the culture medium.

The metabolites were assayed in the culture supernatant, after centrifugation at 13 000 rpm for 5 minutes. The glucose, glycerol, ethanol, pyruvate, succinate, acetate and α-ketoglutarate concentrations were determined by high performance liquid chromatography (HPLC) using an HPX-87H column (Bio-Rad).

The concentrations of volatile compounds (higher alcohols and esters) were determined by gas chromatography.

Results

The I-4216 strain has a maximum fermentation rate (Vmax) greater than that of I-4215, and consumes glucose more rapidly (FIG. 3).

Table 1 below gives the carbon and redox balance of the I-4215 and I-4216 strains. As emerges from the examination of FIG. 3, the most striking result concerns the I-4216 strain, which finishes its fermentation 200 hours earlier than the control strain. Since the adapted strain shows a final biomass that is less than that of the control strain (table 1), the specific fermentation rate is therefore greatly increased compared with that of I-4215

TABLE 1

| Compound (g/l) | I-4215 | I-4216 |
|---|---|---|
| Glucose | 239 ± 2 | 239 ± 2 |
| Biomass | 2.1 ± 0.2 | 1.3 ± 0.2 |
| Ethanol | 119 ± 2.5 | 118 ± 4.0 |
| Glycerol | 7.4 ± 0.3 | 7.3 ± 0.7 |
| Pyruvate | 0.1 ± 0.0 | 0.1 ± 0.0 |
| Acetate | 0.70 ± 0.06 | 0.35 ± 0.01 |
| 2,3-Butanediol | 0.4 | 0.6 |
| Succinate | 1.0 ± 0.1 | 0.9 ± 0.1 |
| Carbon balance (%) | 101 | 100 |
| Redox balance (%) | 103 | 101 |

Furthermore, the adapted strain produces less acetate than I-4215. This reduction reaches a factor of 2 (FIG. 3; table 1).

Since acetate is the second source of NADPH after the PPP, the decrease in acetate production suggests that the PPP flux is increased in these strains.

The obtaining of a strain with low acetate production is of interest in enology, in particular in highly clarified white must wine-making processes.

No marked effect is observed on the production of the other by-products. The carbon and redox balances are equilibrated in the two strains (table 1).

The production of aromatic compounds was analyzed by gas chromatography. The results obtained on aroma production are summarized in table 2 below.

TABLE 2

| | I-4215 | I-4216 |
|---|---|---|
| isobutanol | 24.5 ± 1.4 | 29.5 ± 1.2 |
| isoamyl alcohol | 280.6 ± 13 | 415.4 ± 6.4 |
| 2-phenylethanol | 90.2 ± 3.9 | 195.8 ± 0.6 |
| isoamyl acetate | 0.0 ± 0.0 | 12.4 ± 0.2 |

Marked effects were observed on aroma production by the adapted strain compared with the control strain (I-4215).

The I-4216 adapted strain exhibits an increased production of isoamyl alcohol and isoamyl acetate (1.5 times greater than EC1118) (table 2). Furthermore, the production of 2-phenylethanol is higher in this strain (twice as high) (table 2). The phenylethanol is produced from the degradation of phenylalanine. This amino acid is synthesized from an intermediate of the PPP, erythrose-4-phosphate. An increase in this compound is consistent with an increase in PPP flux.

A considerable modification of the aromatic properties is thus observed, partly explained by an increase in PPP flux.

These modifications are of great interest in enology since isoamyl acetate and 2-phenylethanol are considered to be positive aromas of wines and are associated with banana and pear flavors for the first and rose and flower flavors for the second.

Quantification of pentose phosphate pathway flux in the I-4216 adapted strain

All the modifications observed show that the pentose phosphate pathway flux has been amplified in the I-4216 adapted strain. For the purposes of confirming and quantifying this effect, a comparative ¹³C flux analysis in I-4216 and I-4215 was carried out.

Culture Conditions

The cultures are performed in penicillin vials containing 10 ml of synthetic medium, the composition of which is modified so that it contains 100 g/l of glucose of which 40% is labeled with ¹³C on Cl (Eurisotope) and 1.4 g/l of NH₄Cl as sole carbon source.

The vials are inoculated at 0.01 ODU.

Cell Treatment

At OD 3 (middle of the exponential growth phase), the cells are centrifuged. The incorporation of ¹³C into the amino acids is determined after acid hydrolysis of the biomass and derivatization as described by Gombert et al. (2001). The culture supernatant is preserved with the aim of determining the production of extracellular by-products by HPLC.

Analytical Method

Gas chromatography coupled to mass spectrometry (GC-MS) was used to determine the carbon isotope distribution in the proteinogenic amino acids obtained during the cultures performed on ¹³C glucose. The method used is that described by Christensen and Nielsen (1999). The analysis is carried out using GC-MS of GC-17A/GCMS-QP5050A type (Shimadzu) operating in EI (Electron Impact) ionization mode and at 70 eV. The column used during the analyses is a DB-1701 column (length=30 mm, ID=250 μm, film=0.10 μm) supplied by Agilent. The mass spectrometer operates in SIM mode.

Data analysis and Processing

From the raw data obtained from the measurements by GC-MS, the ¹³C incorporation rate of each fragment is calculated in the following way: SFL=100×[(1.m₁+2.m₁ + . . . + n.mₙ)×(m₀+m₁+m₂+ . . . + mₙ)⁻¹], m_{i>0} represents the abundance of the molecules containing i labeled carbons.

Furthermore, the concentration of extracellular metabolites produced is assayed by HPLC.

All these data (SFLs and extracellular metabolites) were used as constraint of the mathematical model (Gombert et al., 2001) to quantify the fluxes in carbon metabolism. 50 repeats are carried out and the flux distributions represented in FIG. 4 are the mean of 25 selected distributions.

Results

The flux distribution, estimated in the exponential growth phase, shows a considerable increase in PPP flux for the I-4216 strain. At this stage, a slight decrease in ethanol production is observed (168 and 165 mmol/100 mmol glucose for I-4215 and I-4216, respectively), while the flux directed toward the biomass shows an overall decrease in the I-4216 strain (FIG. 4). The loss of carbons resulting from the amplification of the PPP flux in the ECA5 strain therefore appears to be compensated for by a decrease both in biomass and in ethanol.

Balance for I-4216 strain fermentation on a Chardonnay must

Culture Conditions:

100-liter fermenters were inoculated with one million cells per ml, from a preculture in liquid levain of the I-4215 and I-4216 strains. The media used for the fermentation and for the preculture are the same (chardonnay must harvested in 2007). An oxygenation of 15 mg/l and also an addition of diammonium hydrogen phosphate (30 g/hl) were carried out after the maximum rate.

Analytical Methods:

Assaying of ethanol: assaying with a Paar densitometer. This method consists in distilling the wine alkalinized with a suspension of calcium hydroxide (98%) and in measuring the density of the distillate. For this measurement, the distillate is directly injected into an oscillating system, the vibration frequency of which is modified by the weight of the substance injected.

Measurement of volatile acidity: after distillation by nitrogen entrainment at 98° C., the acids of the wine are brought into contact with a colored reagent (bromophenol blue), the coloring intensity variations of which are proportional to the content of volatile acidity of the wine.

Measurement of aroma compounds: the concentrations of volatile compounds (higher alcohols and esters) were determined by gas chromatography on an Agilent Headspace 6890 system equipped with an HP 7694 sampler and an FID detector.

As previously observed, I-4216 produces less volatile acidity (including acetate) than I-4215 (4 times less) (table 3), and produces much more aromatic compounds such as isoamyl acetate (4 times more) (table 4).

A decrease in ethanol production in the I-4215 strain is also observed. It is reflected by a decrease in its ethanol yield from 0.47 to 0.46 g of ethanol/g of sugar consumed (table 3).

TABLE 3

| | | | | | | Ethanol |
|---|---|---|---|---|---|---|
| | | | | | | yield g of |
| | | | Volatile | | Glucose + | ethanol/g |
| | | Acidity | acidity | | fructose | of sugar |
| Sample | pH | g/l H₂SO₄ | g/l H₂SO₄ | Alcohol % vol | g/l | consumed |
| I-4215 | 3.3 ± 0.0 | 3.9 ± 0.1 | 0.4 ± 0.0 | 14.1 ± 0.0 | 0.8 ± 0.0 | 0.47 ± 0.00 |
| I-4216 | 3.3 ± 0.0 | 3.7 ± 0.1 | 0.1 ± 0.0 | 14.0 ± 0.0 | 1.6 ± 0.0 | 0.46 ± 0.00 |

TABLE 4

| | I-4215 | I-4216 |
|---|---|---|
| isobutanol | 22.5 ± 0.7 | 32.23 ± 1.5 |
| isoamyl alcohol | 207.3 ± 6.9 | 250.4 ± 8.8 |
| isobutyl acetate | 0.0 ± 0.0 | 0.5 ± 0.0 |
| isoamyl acetate | 7.2 ± 0.1 | 33.6 ± 1.9 |
| ethyl butyrate | 0.3 ± 0.0 | 0.4 ± 0.0 |
| ethyl hexanoate | 0.3 ± 0.0 | 1.5 ± 0.1 |

### References

Becker, J. and E. Boles (2003). “A modified Saccharomyces cerevisiae strain that consumes L-Arabinose and produces ethanol”. Appl Environ Microbiol 69(7): 4144-50.

Blank, L. M., F. Lehmbeck, et al. (2005). “Metabolic-flux and network analysis in fourteen hemiascomycetous yeasts.” FEMS Yeast Res 5(6-7): 545-58.

Christensen, B. and J. Nielsen (1999). “Isotopomer analysis using GC-MS.” Metab Eng 1(4): 282-90.

Eliasson, A., C. Christensson, et al. (2000). “Anaerobic xylose fermentation by recombinant Saccharomyces cerevisiae carrying XYL1, XYL2, and XKS1 in mineral medium chemostat cultures.” Appl Environ Microbiol 66(8): 3381-6.

Gombert, A. K., M. Moreira dos Santos, et al. (2001). “Network identification and flux quantification in the central metabolism of Saccharomyces cerevisiae under different conditions of glucose repression.” J. Bacteriol 183(4): 1441-51.

Jeppsson, M., B. Johansson, et al. (2002). “Reduced oxidative pentose phosphate pathway flux in recombinant xylose-utilizing Saccharomyces cerevisiae strains improves the ethanol yield from xylose.” Appl Environ Microbiol 68(4): 1604-9.

Pitkanen, J. P., E. Rintala, et al. (2005). “Xylose chemostat isolates of Saccharomyces cerevisiae show altered metabolite and enzyme levels compared with xylose, glucose, and ethanol metabolism of the original strain.” Appl Microbiol Biotechnol 67(6): 827-37.

Saint-Prix, F., L. Bonquist, et al. (2004). “Functional analysis of the ALD gene family of Saccharomyces cerevisiae during anaerobic growth on glucose: the NADP+-dependent Ald6p and Ald5p isoforms play a major role in acetate formation.” Microbiology 150(Pt 7): 2209-20.

Sonderegger, M., M. Jeppsson, et al. (2004). “Fermentation performance of engineered and evolved xylose-fermenting Saccharomyces cerevisiae strains.” Bioeng 87(1): 90-8.

1. A method for obtaining mutant yeast strains having an amplified PPP, characterized in that it comprises culturing the strains on a substrate based on a gluconic acid salt.

2. The method as claimed in claim 1, characterized in that the culturing of the strains is carried out over at least 10 generations, at an incubation temperature of from 16° C. to 32° C., preferably of 28° C., with shaking.

3. The method as claimed in claim 1, characterized in that it also comprises a step of selecting the adapted strains having accumulated mutations allowing amplification of the PPP, by means of a growth test, said strains having an OD greater than at least 1.3 times that of the parental strain.

4. Yeast strain mutants which have an amplified PPP compared with a parental strain, characterized in that they are capable of being obtained by means of a method of culture comprising the steps
of adapting a yeast strain, over at least 10 generations, using as sole carbon source a substrate based on gluconate,
of selecting the mutants having accumulated mutations allowing amplification of the PPP, having a final OD at least 1.3 times greater than that of the parental strain not subjected to the above adaptation conditions.

5. The mutants as claimed in claim 4, characterized, compared with a parental strain, by an amplified PPP, a greater fermentation rate and a reduced acetate production.

6. The mutants as claimed in claim 4, characterized by an increased production, compared with a parental strain, of aromatic compounds, such as isoamyl alcohol, isoamyl acetate or phenylethanol.

7. The mutants as claimed in claim 4, characterized in that they belong to the Saccharomyces genus, in particular to the species cerevisiae, bayanus, uvarum or kudriavzevii, non-Saccharomyces genera, or hybrids.

8. The mutant strain of Saccharomyces cerevisiae deposited at the Collection Nationale de Culture de Microorganismes (CNCM) [French National Microorganism Culture Collection], 25 rue du Dr Roux, Paris, for the purposes of a filing according to the Treaty of Budapest, under No. CNCM 1-4216, on Jul. 29, 2009.

9. A method of fermentation comprising the strains as claimed in claim 4.
